(19) United States
(12) Patent Application Publication
(10) Pub. No.: US 2006/0111684 A1
(43) Pub. Date: May 25, 2006

(54) Disposable absorbent article having viewable indicia

(76) Inventors: Maria Luisa M. Berba, Quezon City (PH); Ana Maria R. Marcelo, Antipolo Rizal (PH); Maria Socorro F. Medina, Cainta (PH); Paulo Roberto Amaral Serra, Sao Paulo (BR); Jenny G. Du, Chalfont, PA (US); Tarun K. Arora, Edison, NJ (US); Luiz Henrique Rachid, Sao Jose dos Campos (BR); Francisco Antonio Rimoli, Mogi das Cruzes (BR)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/262,048

(22) Filed: Oct. 28, 2005

Related U.S. Application Data
(60) Provisional application No. 60/623,030, filed on Oct. 28, 2004.

(51) Int. Cl. A61F 13/15

(52) U.S. Cl. 604/361; 604/385.01

(57) ABSTRACT

An absorbent article including a first layer having an internal surface and an external surface, a second layer having an internal surface and an external surface, indicia arranged on at least one of the internal surfaces of the first layer the second layer, at least one of the first layer or the second layer having a light transmittance of at least 45% so that the indicia is viewable from at least one of the external surfaces.

### CROSS-REFERENCE TO RELATED APPLICATION

The present application claims priority of U.S. Provisional Patent Application Ser. No. 60/623,030 filed Oct. 28, 2004.

### FIELD OF THE INVENTION

The present invention relates to absorbent articles. More particularly, the present invention relates to an absorbent article having indicia printed on an internal surface of the article, the indicia being substantially viewable through an entire outer surface of the article.

### BACKGROUND

Absorbent articles to be attached to a crotch portion of a woman's undergarment such as pantiliners, sanitary napkins and incontinence pads, are well known in the art. Conventional sanitary protection articles of this type are intended to absorb and retain menstrual fluid such that the fluid does not leak out of the article.

In recent years, a growing number of users have chosen to wear absorbent articles, in particular pantiliners, even when the amount of vaginal discharge is relatively small. Such users do not wear pantiliners primarily for absorption of menstrual fluid, but rather wear the liner for stain prevention of undergarments. By using pantiliners in this manner, the crotch portion of the panties is prevented from staining so that the panties retain a cleaner, newer look.

When pantiliners are worn when vaginal discharge is small wearers consider comfort to be of prime importance. Therefore, lighter, thinner and smaller pantiliners have become popular. Examples of such pantiliners may be found in U.S. Appl. No. 20030114822 (Collado et a.) and U.S. Pat. No. 6,440,111 (Berba et al.), the disclosures of which are incorporated herein in their entirety.

When pantiliners are worn on a daily basis women may want to select a pantiliner having a particular color to match their garment and/or according to their mood. Thus, some known liners have been provided with colored patterns. For example, U.S. Pat. No. 4,623,340 (Luceri) provides an absorbent article having an interior layer in a face-to-face relationship with the interior surface of the cover. The interior layer is relatively dark colored and preferably a thermoplastic material. When the outer and interior layers are co-embossed, the embossed area provides a color contrast with the un-embossed area.

Others have attempted to provide graphics or designs on absorbent articles. For example, US 2004/0015145 (Miura et al.) purports to disclose an absorbent article having a graphic visible through the body-contacting surface. The garment-facing layer has a graphic printed on the body-facing surface. The graphic is visible around the perimeter region of the absorbent article.

WO 03/053313 (Criston et al.) purports to disclose an absorbent article that is provided with a colored portion viewable from the top surface of the absorbent article which gives a perception of depth within the absorbent article. The creation of depth perception is purportedly accomplished by the use of at least two shades of a color.

Dark pantiliners such as those disclosed in U.S. Pat. No. 6,586,653 (Graeme et al.) and WO 02/07661 (Gagliardi et al.) are also commercially available. These pantiliners have a dark color such as black, the dark color purportedly minimizing the discolorization of the absorbent article when body fluids including vaginal discharge and menstrual fluid are absorbed.

Known colored absorbent articles have inherent limitations and drawbacks which the present invention attempts to overcome. In particular, there is still a need for an absorbent article having graphics or indicia printed on an internal surface of the article, the indicia or graphics being visible through an entire outer surface of the article. Such an article would appeal to users, especially those who coordinate their absorbent articles to match their undergarments or moods.

### SUMMARY OF THE INVENTION

According to a first aspect of the invention, the present invention provides an absorbent article including a first layer having an internal surface and an external surface, a second layer having an internal surface and an external surface, indicia arranged on at least one of the internal surface of the first layer or the internal surface of the second layer, and at least one of the first layer or the second layer has a light transmittance of at least 45% so that the indicia is viewable from at least one of said external surfaces.

According to a second aspect of the invention, the present invention provides an absorbent article including a liquid permeable cover layer having an internal surface and an external surface, a liquid impermeable barrier layer having an internal surface and an external surface, indicia arranged on the internal surface of said barrier, the cover layer having a light transmittance of at least 45% over its entire surface area and the indicia having a ΔE of at least 9 as measured from the external surface of said cover layer.

According to a third aspect of the invention, the present invention provides an absorbent article including a liquid permeable cover layer having an internal surface and an external surface, a liquid impermeable barrier layer having an external surface, an internal layer arranged between the cover layer and the barrier layer, the internal layer having a first surface in abutting relationship to the internal surface of said cover, indicia arranged on said first surface of said internal layer, the cover layer having a light transmittance of at least 45% over its entire surface area and said indicia having a ΔE of at least 9 as measured from the external surface of said cover layer.

According to a fourth aspect of the invention, the present invention provides an absorbent article including a cover layer having an internal surface and a external surface, a barrier layer having an internal surface and a external surface, the barrier having non-colored portion and a colored portion printed on the internal surface of the barrier layer; the cover layer having a light transmittance of at least 45% over its entire surface area and the colored portion having a ΔE of at least 9 as measured from the external surface of said cover layer.

According to a fifth aspect of the invention, the present invention provides an absorbent article including a liquid permeable cover layer having an internal surface and an external surface, a liquid impermeable barrier layer having an external surface, an internal layer arranged between said cover layer and said barrier layer, the internal layer having a first surface in abutting relationship to said internal surface of the cover and a second surface in opposed relationship to the first surface, indicia arranged on the second surface of said internal layer, the cover layer and the internal layer having a light transmittance of at least 45% over their entire surface area and the indicia having a ΔE of at least 9 as measured from the external surface of said cover layer.

### BRIEF DESCRIPTION OF THE FIGURES

Examples of embodiments of the present invention will now be described with reference to the drawings, in which:

FIG. 1 is a perspective view of an absorbent article according to the present invention;

FIG. 2 is an exploded perspective view of the absorbent article shown in FIG. 1;

FIG. 3 is an exploded perspective view of the absorbent article shown in FIG. 1, showing another embodiment thereof;

FIG. 4 is an exploded perspective view of the absorbent article shown in FIG. 1, showing yet another embodiment thereof;

FIG. 5 is a sectional view of the absorbent article shown in FIG. 4;

FIG. 6 is an exploded perspective view of an absorbent article according to another embodiment of the present invention;

FIG. 7 is a is an exploded perspective view of an absorbent article according to still another embodiment of the present invention.

### DETAILED DESCRIPTION OF THE INVENTION

The present invention relates to “disposable absorbent articles”, and in particular, a disposable absorbent article having at least two layers and indicia printed on an “internal surface” of one of the layers, the indicia being viewable by the naked eye when viewing at least one external surface of the article. Preferably, the indicia is visible “through an entire outer surface of the article” (as defined below). In a preferred embodiment of the present invention the disposable absorbent article is a pantiliner.

“Disposable absorbent articles” such as, pantiliners, sanitary napkins, interlabial devices, adult incontinent devices and diapers are well known in the art. These articles typically have a fluid permeable body-facing side and fluid impermeable garment facing side. Additionally, such articles may include an absorbent core for retaining fluids. Disposable absorbent articles are intended to be discarded after use, i.e. not laundered or otherwise reused.

The term “internal surface” or “inner surface” as used herein includes the internal surface of the body facing layer (i.e. non-body facing surface), the internal surface of the barrier layer (i.e. the non-garment facing surface), and/or either surface of a layer arranged between the body facing layer and the barrier layer.

The term “body facing layer” is used herein interchangeable with the term “cover” and the term “garment facing layer” is used herein interchangeable with the term “barrier.”

As used herein, the terms “drapeable” and “drapeability” are used interchangeably and mean having a flexural resistance of about 40 g or less as tested by the Modified Circular Bend Test, ASTM 4032-82. Drapeable articles of the present invention have a flexural resistance of preferably about 40 or less, more preferably about 30 or less, and most preferably about 20 or less.

As used herein, the term “indicia” refers to a color, an image or a design. The indicia of this invention may be any color other than white. Alternately, the indicia may be present as an image or design. The image or design may be a symbol, figure, line, character or similar color-containing graphic that depicts a color difference from the substrate that the indicia is printed on. The indicia can provide an emotional benefit when the user views the absorbent article. The indicia may include multiple colors (i.e., at least two colors) and may include flowers, swirls, dots, butterflies, stars, stripes, lines, squares, suns, zig-zags, animals, logos, cartoon characters, seasonal icons, and other popular symbols. As used herein, the term discrete shall mean distinct, unconnected, discontinuous element or entity. For example, a butterfly made from two colors may be considered a discrete element. The phrase “discrete pattern” therefore, shall mean a pattern of discrete entities. A series of butterflies spaced apart shall be considered a discrete pattern and may make up the indicia.

When the terminology “viewable over the entire external surface” or the like is used herein it does not require that the indicia or the like be printed over an entire internal surface of the article. Rather, this terminology means that the article does not contain any structure that would prevent the indicia from being viewed from the external surface of the article if indicia was printed on an internal surface in such area of the article. Stated another way the material(s) of the article arranged between the indicia and the relevant outer surface of the article from which the indicia is being viewed has a light transmittance of at least 45% over the material's entire surface area. In this manner, no matter where the indicia is arranged, the material located between the indicia and the external surface of the article being viewed will not prevent the indicia from being readily visible.

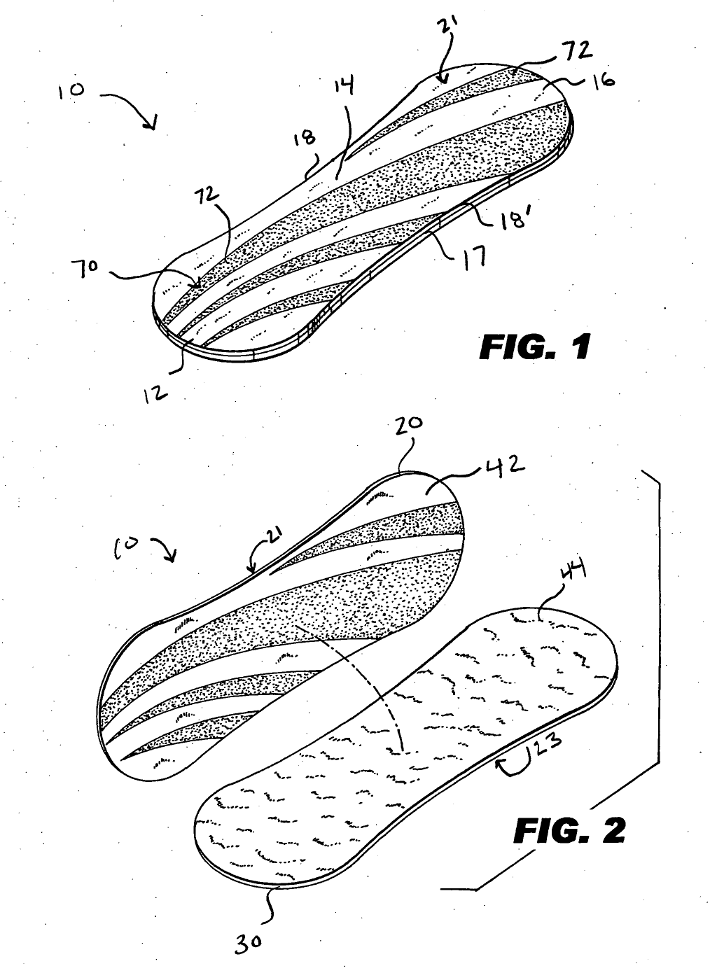

Reference is made to FIGS. 1 and 2, which depict an absorbent article 10 according to a first embodiment of the present invention. The absorbent article 10 includes a body facing layer 20 having a body facing surface 21, and an inner surface 42. The absorbent article 10 further includes a garment facing layer 30 having garment facing surface 23 and an internal surface 44. In the absorbent article 10, indicia 70 in the form of colored lines 72 is printed on the inner surface 42 of a body-facing layer 20 (cover). In the embodiment shown in FIGS. 1 and 2, the portions of the body-facing layer 21 that are not colored, i.e. the portions of the body-facing layer outside the colored lines 72, are white. In the embodiment shown in FIGS. 1 and 2, the garment facing layer (barrier layer) 30 is entirely non-colored, i.e white.

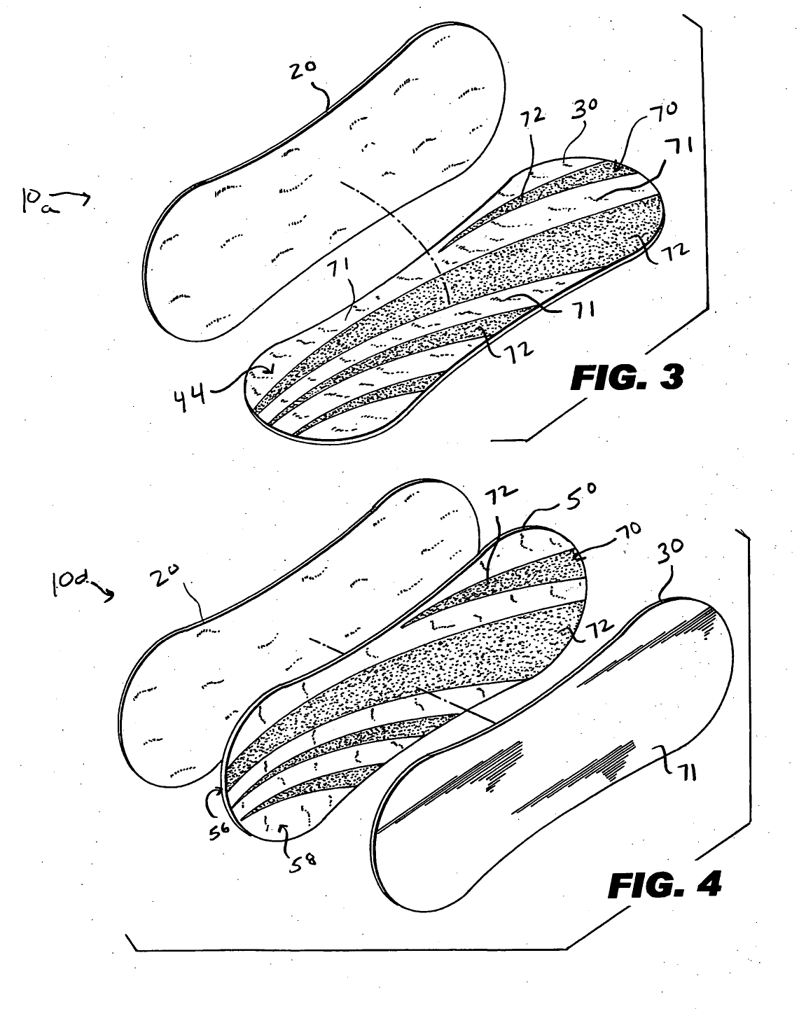

Reference is made to FIG. 3 which shows another embodiment of the present invention 10a. In this embodiment the internal surface 44 of garment facing layer 30 is provided with indicia 70 in the form of colored lines 72 and the remainder of the garment facing layer 30 is white. Thus the barrier 30 includes a colored portion, i.e. indicia 70, and a noncolored portion 71.

In another embodiment of the invention 10b shown in FIG. 6, the body-facing layer 20 is embossed with a pattern of birds 90. The body-facing layer 20 may be embossed to include other designs such as flowers. Indicia 70 in the form of colored lines 72 is printed on inner surface 44 of the garment facing layer 30 (barrier). Thus the barrier 30 includes a colored portion, i.e. indicia 70, and a noncolored portion 71.

In another embodiment of the invention 10c in FIG. 7, the indicia 70 is printed to the internal surface 44 of the barrier 30, and the indicia 70 includes colored lines 72 having a first color (e.g. green) and a bird 92 having a second color (e.g. blue). Thus the indicia 70 includes at least two colors. Other designs are also possible, for example a butterfly having a body with a first color and wings with a second color. Thus, in the embodiment shown in FIG. 7 the barrier includes a first colored portion 72, a second colored portion 92, and a non-colored portion 71.

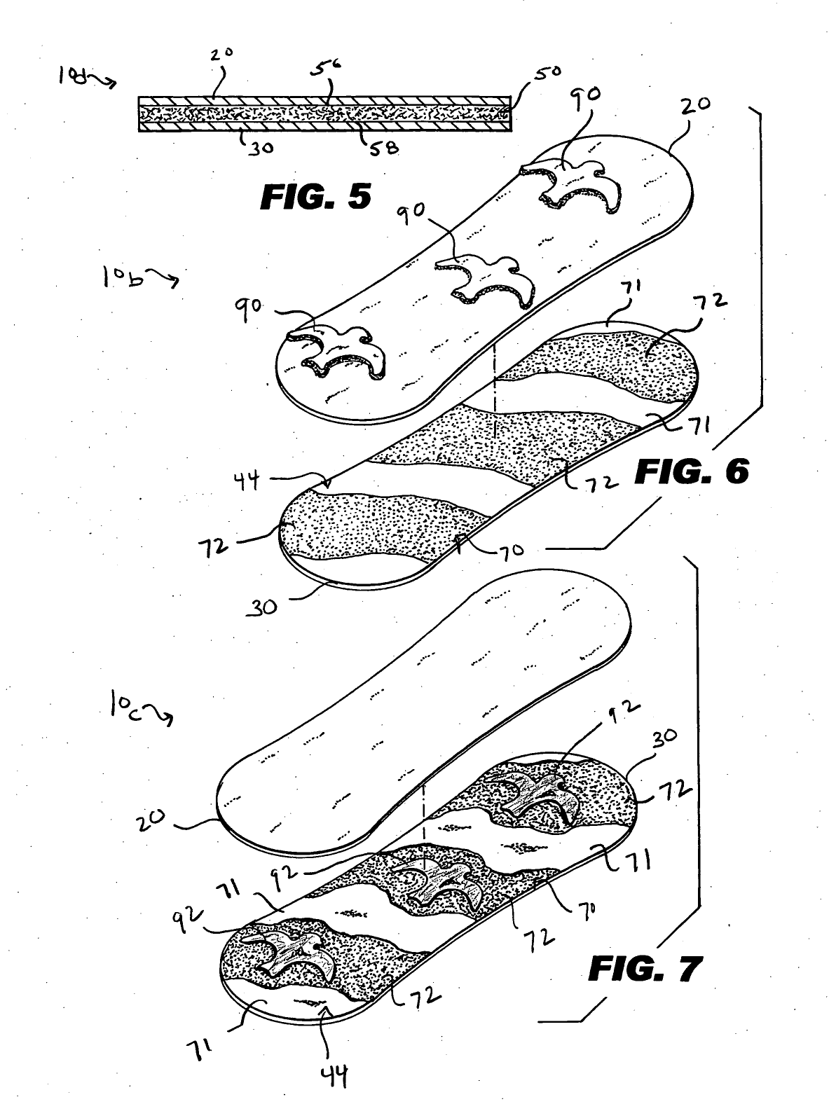

Another embodiment of the present invention 10d is shown in FIG. 4, in this embodiment the article includes a body-facing layer 20, a garment facing layer 30 and an absorbent core 50. Indicia 70 in the form of colored lines 72 are printed on a bottom surface 58 of the core 50. Alternatively the indicia may be printed on the top surface 56 of the absorbent core 50. In the embodiment shown in FIGS. 4 and 5, the barrier 30 is not colored, i.e. the entire barrier 30 is a non-colored portion 71. Surfaces 56 and 58 are shown in FIGS. 4 and 5. Additionally, the indicia may be printed on either or both surfaces of a transfer layer arranged between the cover and a core structure, if a transfer layer is used.

In the present invention, the indicia is printed on an internal surface of the absorbent article. Thus, in order to view the indicia from an external surface of the article, the layers of the article located between the internal surface upon which the indicia is printed and the external surface of the article being viewed must have a sufficient transparency.

As used herein, the term “sufficient transparency” means a light transmittance level of at least 45%. It has been found that in order for indicia printed on an internal surface to be visible from an external surface of the article, the material layer arranged between the indicia and the external surface must have a light transmittance level of at least 45%. If multiple layers are arranged between the indicia and the external surface then the layers must have a combined light transmittance level of at least 45%. For example, if an absorbent article is made from a cover and a barrier and has a series of stars printed on the internal facing surface of the barrier, in order for the stars to be viewable from the body facing surface of the article, the cover must have a light transmittance of at least 45%. Preferably, according to the present invention, the cover has a light transmittance over its entire surface area of at least 45%. In this way, no matter where the indicia is arranged it will be viewable from the body facing surface of the article. Similarly, in order for the indicia to be viewable from the garment facing surface, the barrier must have a light transmittance of at least 45%. Preferably, according to the present invention, the barrier has a light transmittance over its entire surface area of at least 45%. In this way, no matter where the indicia is arranged it will be viewable from the garment facing surface of the article. Preferably, both the cover and the barrier have a light transmittance of at least 45% such that the indicia is viewable from both the body facing surface of the article and the garment facing surface of the article.

If, for example, an absorbent article according to the present invention is made from a cover, barrier, and an absorbent core arranged between the cover and the barrier, and has a series of stars printed on the internal facing surface of the barrier, in order for the stars to be viewable from the body facing surface of the article, the cover and absorbent core combined must have a light transmittance of at least 45%. If the stars are being viewed from the barrier side, then the barrier must also have a light transmittance of at least 45%. In preferred embodiments of the present invention the cover and core have a combined light transmittance of at least 45% over their entire combined surface area and the barrier has a light transmittance of at least 45% over its entire surface area.

The above examples are provided for illustrative purposes only but other embodiments are possible within the scope of the invention so long as the layers of the article that are arranged between the indicia and the surface of the article being viewed have a total light transmittance of at least 45% (either singularly if one layer or combined if more than one layer).

As used herein, the term “color” or “colored”, when referring to the indicia or colored portion of the article, includes any color other than white. Colors can be measured according to an internationally recognized L Hunter Scale. This system is based on three variables L*, a*, b*.

A complete technical description of the system can be found in an article by R. S. Hunter, “Photoelectric Color Difference Meter”, Journal of the Optical Society of America, Vol. 48, pp. 985-995, 1958. Devices specially designed for the measurement of color on the Hunter scales are described in U.S. Pat. No. 3,003,388 to Hunter et al., issued Oct. 10, 1961. In general, Hunter Color “L” scale values are units of light reflectance measurement, and the higher the value is, the lighter the color is since a lighter colored material reflects more light. In particular, in the Hunter Color System, the “L” scale contains 100 equal units of division, absolute black is at the bottom of the scale (L=0) and absolute white is at the top of the scale (L=100). When color is defined according to this system, L* represents lightness (0=black, 100=white), a* and b* independently each represent a two color axis, a* representing the axis red/green (+a=red, −a=green), while b* represents the axis yellow/blue (+b=yellow, −b=blue).

When comparing colors it is useful to calculate the function E for each color where E²=L²+a*²+b*². The difference in color between two adjacent surfaces or two adjacent colors may be represented as follows:
ΔE=[(L*₂−L*₁)²+(a*₂−a*₁)²+(b*₂−b*₁)²]^{1/2}

For purposes of the present invention, as discussed in further detail below, the uncolored portion of the barrier of the inventive samples, i.e. the white portion, was used to determine L*₂, a*₂, b*₂, and the colored portion of the barrier as measured through the cover layer was used to determine L*₁, a*₁, and b*₁. ΔE was then calculated from these values.

Color measurements required to calculate ΔE can be made using a tristimulus colorimeter (Minolta Chromameter® CR300) using the CIE (International Commission on Illumination) L*a*b* color coordinate standard.

The term “uncolored”, “non-colored”, “white” or the like, as used herein, means those colors having an L* value of at least 90, an a* value equal to 0±2, and a b* value equal to 0±2.

For colors to be distinctly seen from the substrate on which they are printed, a single color should have a difference in color from the substrate (or reference) that it is compared against. For example, a red dot printed on white paper will have a distinct ΔE.

It has been found in accordance with the present invention that when the layers of material arranged between the indicia and the external surface being viewed have a light transmittance of at least 45% then the indicia will be readily visible to the naked eye provided that the measured ΔE of the indicia is at least 9.0 when measured from the external surface being viewed.

Turning to FIG. 1 the absorbent article according to the present invention generally include an anterior portion 12, a posterior portion 16 and two longitudinal edges 18, 18′ which connect the anterior portion 12 and posterior portion 16. The outer perimeter of absorbent article forms the silhouette 17. In the embodiment shown in FIG. 1, the longitudinal edges 18, 18′ are slightly concave and the two ends are substantially equivalent in width. Other embodiments are possible. For example, the two longitudinal edges may decrease in width toward the direction of one end. An absorbent article having this shape may be suitable for thong-type underwear.

Absorbent article 10 as shown in FIG. 1 has three portions: an anterior portion 12, a central portion 14, and a posterior portion 16 and at least a body-facing layer 20 and a garment-facing layer 30. In one embodiment of the present invention, the absorbent article does not contain an absorbent core.

In one embodiment of the present invention, the absorbent article has a body-facing layer and a garment-facing layer. In another embodiment, the absorbent article additionally includes an absorbent layer arranged between the body-facing layer the garment-facing layer. Other embodiments may include additional layers such as, a transfer or distribution layer, multiple layer absorbent layers and unitized versions of two or more layers.

The absorbent article 10 of the present invention is intended to manage low volumes of fluid encountered both menstrually and intermenstrually, i.e, daily. Preferably, the absorbent core of the various embodiments of the present invention have less than 0.1 grams of absorbent fibers, and the absorbent article has the ability to absorb 6.0 grams or less of fluid.

In another embodiment, the absorbent article is drapeable.

The silhouette of absorbent articles of this invention includes those designed to fit garments having conventionally-shaped crotches, e.g., briefs and bikinis. Additionally, absorbent articles of the present invention may also be designed to fit garments having abbreviated crotches including thong, string underwear, G-string, Rio cut, Brazilian cut, etc.

Body-Facing Layer

The absorbent article of the present invention includes a liquid permeable layer also referred to as a body-facing layer or cover 20 (shown FIG. 2). The exterior of the body-facing layer forms the body-facing surface 22 of the absorbent article. The body-facing layer may be a single layer or be made from multiple layers. The body-facing layer may be formed from any fluid pervious material or combinations of materials that are comfortable against the skin and permits fluid to penetrate. For instance, the body-facing layer may be a fibrous non-woven fabric made of fibers or filaments of polymers, such as polyethylene, polypropylene, polyester, or cellulose, and combinations thereof. Alternatively, the body-facing layer may be formed from an apertured polymeric film.

For example, any material with cloth-like features may be used for the body-facing layer. Such material includes nonwoven, such as spunlace, woven, and knitted materials. In particular, spunlace material may be made from about 0 to about 100% rayon and from about 0 to about 100% polyester. The spunlace material may also be made from about 10 to about 65% rayon and from about 35 to about 90% polyester may be used. Optionally, the material used for the body-facing layer may include binders, such as thermoplastic binder fibers and latex binders.

In one embodiment, the body-facing layer 20 is a single sheet of material having a width sufficient to form the body-facing surface of the absorbent article. In another embodiment, the body-facing layer has at least two layers.

The body-facing layer, whether a single layer or multiple layers, may have absorbent capabilities, i.e., retains fluid. If a separate absorbent layer is used, the body facing layer may be longer and wider than the absorbent core or be of similar size as the absorbent core.

In another embodiment, the absorbent article 10 of the present invention includes a body-facing layer 20 overlaying an absorbent core structure 50 (shown in FIG. 5). The body-facing layer 20 includes a body-facing surface 22 and a inner surface 42. The body-facing layer 20 may be formed from any fluid pervious material that is generally compliant, soft feeling, and non-irritating to the user's skin and permits fluid to penetrate to the absorbent core, which retains the fluid. The body-facing layer 20 generally functions to transport fluid away from the wearer into the absorbent article. In this manner, fluid and moisture are removed from contacting the wearer, thus making the wearer feel dry and comfortable. In addition to transporting fluid, the body-facing layer 20 may also absorb and/or retain fluid as well.

Suitable materials that can be used as the body-facing layer 20 are woven and nonwoven fabrics formed fibers or filaments of cellulose, polyester, polyethylene, polypropylene, nylon, rayon fibers and mixtures thereof or the cover layer may be an apertured thermo-plastic film and formed films. Other materials used in making body-facing layer 20 include gauze or any known porous material with a suitable body contacting surface, including, but not limited to nonwoven webs, plastic nets, and the like. The body-facing layer 20 could also be made from a fibrous nonwoven composite of bicomponent fibers and pulp fluff.

Bicomponent fibers are known in the art and are composed of two polymers with different melting points. At least a portion of the outer surface of each bicomponent fiber has the lower melting polymer. The two polymers may be arranged such that a cross-section of the fiber shows the two polymers in a side-by-side array. Alternatively, the polymers may be positioned in a so-called sheath/core arrangement, in which a core of higher melting polymer is surrounded by a sheath of lower melting polymer.

The body-facing layer 20 may be made by a process of bonding a nonwoven conjugate fiber web in which heated air, which is sufficiently hot to melt one of the polymers of the multicomponent fibers, is forced through the web. This process is referred to as through air bonding. The melting and resolidification of the polymer provides the bonding between the fibers to integrate the web. The air velocity is typically between 100 and 500 feet per minute (30 and 152 m/min) and the dwell time may be as long as 6 seconds. Through air bonding has relatively restricted variability and since through-air bonding requires the melting of at least one component to accomplish bonding, it is particularly useful in connection with webs of conjugate fibers or those which include an adhesive. In the through-air bonder, air having a temperature above the melting temperature of at least one of the exposed components is directed through the web and into a perforated roller supporting the web. Alternatively, the through-air bonder may be a flat arrangement wherein the air is directed vertically downward onto the web. The operating conditions of the two configurations are similar, the primary difference being the geometry of the web during bonding.

A useful bicomponent fiber is a 2.0 denier, 45 mm long staple fiber made of a polyester core and a high density polyethylene sheath. Similar fibers (polyethylene sheath and polypropylene core) are available as Danaklon ES-C or ES Bico (Danaklon A/S, Varde Denmark) or ES Fiber GuangZhou Co. Ltd. (Chisso).

The body-facing layer 20 may optionally be treated with surfactant to manipulate the hydrophobicity/hydrophilicty thereof to facilitate optimal fluid transport properties. The fibers or other materials that make up the body-facing layer 20 should not collapse or lose their resiliency when subjected to body fluid. The fibers may be oriented by a carding process and thermally bonded via embossing. The fiber or filament can be single denier or multidenier.

As previously stated, the body-facing layer may be a single layer or be made from multiple layers. The thickness of the cover may vary from about 0.001 inch (0.025 mm) to about 0.200 inch (5.000 mm), depending on the material chosen. The weight of the body facing layer material preferably is between about 5 and about 150 gsm, more preferably between 15 and 50 gsm and most preferably between 25 and 35 gsm. The body-facing material may be though air bonded. Low denier fibers are preferred to form the fiber web and the temperature of the hot air may be adjusted to bond only the material having the lower melting point. This creates a softer and loftier fiber web.

In another embodiment, the body-facing layer has at least two layers and is made of materials described in the core section.

The body-facing layer, whether a single layer or multiple layers, may also retain fluid. If a separate absorbent core layer is used, the body facing layer may be longer and wider than the core or be of similar size as the core.

The body-facing layer 20 may be embossed with shapes within a given area. For example, a series or a number of features, e.g., circles, triangles, squares, lines, honeycomb, diamond, floral, etc. are embossed over the entire length and width of the outer surface of web. Each embossed feature has a major and minor axis extending therethrough, the major axis length being greater or equal to the minor axis length. The embossed features may be in a repetitive pattern. The embossed pattern may contribute to the softness and drapeability of the fluid management article. The embossments may also serve to hold fluid or to transfer fluid to the core. In an embodiment shown in FIG. 5, flower 90 is embossed in a pattern on body-facing layer 20.

According to one embodiment of the present invention indicia 70 is printed on the inner surface 42 of the body-facing layer 20.

Absorbent Core Structure

One embodiment of the present invention includes an absorbent layer or core structure 50, which may be a single layer or, alternately, be made of multiple layers. Absorbent materials used in the absorbent layer may include, but are not limited to, absorbent fibers, such as cellulose fibers, including, but not limited to wood pulp, regenerated cellulose fibers, and cotton fibers, rayon fibers and the like; superabsorbent fibers or particles; other naturally occurring absorbent materials, such as peat moss; and other synthetic absorbent materials, such as foams, and the like. The absorbent layer may also include one or more of the following: thermoplastic binder fibers, latex binder, perfumes, or odor-controlling compounds or compositions. The absorbent layer may be compressed or uncompressed, embossed, or calendered. Additionally, the absorbent core may be made from any known absorbent bicomponent fibers, including those made, for example, from polyester, polyethylene, polypropylene and any combinations thereof. The absorbent material may be woven, nonwoven, or knitted and made by any process. For example the absorbent material may be wet laid, carded, or air laid.

Nonwoven webs are preferred as starting materials to form the absorbent core structure 50. Suitable fibers useful for making such nonwoven webs include polyolefin, polyester fibers, bicomponent fibers, and mixtures thereof. Such fibers may be fusible fibers.

In one embodiment of the invention shown in FIG. 5, the absorbent core structure 50 is a single layer material having a top surface 56 and a bottom surface 58. In the embodiment of the invention shown in FIG. 5, indicia 70 may be printed on at least one of the internal surface 42 of the body facing layer 20, the top surface 56 of core 50 and/or the bottom surface 58 of the core 50. More preferably, the indicia 70 is printed on the internal surface 44 of the barrier 30.

The absorbent core structure 50 of the present invention may contain any known absorbent materials including, but not limited to, absorbent fibers, such as, cellulose fibers, including, but not limited to wood pulp, regenerated cellulose fibers, e.g., rayon and cotton fibers, rayon fibers and the like; other naturally occurring absorbent materials, such as, sphagnum or peat moss; and other synthetic absorbent materials. Pulp fibers may be obtained as IP “SUPERSOFT” ELM supplied by the International Paper Company (Memphis, Tenn.), “RAYFLOC” E-Type Cellulosic Fluff Pulp, (ITT Rayonier), or Korsnas Vigorfluf-EN White (KorsncAs, Gavle, Finland). Georgia Pacific EF-100, or Weyerhauser NB416L, treated or untreated pulp. Additionally, the absorbent core structure 50 may include one or more of the following: binders, such as, thermoplastic and latex, odor-controlling compounds, e.g., perfumes, EDTA (ethylenediaminetetraacetic acid), anti-microbial agents, wetting agents, wetness indicator material, materials for administering or delivering medicaments, such as encapsulated medicaments, and materials for maintaining skin moisture, such as encapsulated moisturizers.

The core may include only materials, such as, a hot melt adhesive containing fluid absorbing polymers. One example of such a material is disclosed in EP 1 013 291 A1, the disclosure of which is herein incorporated in entirety by reference.

A representative, non-limiting list, of absorbent material useful in the absorbent core structure 50 includes, but is not limited to, natural cellulosics, such as, cotton and wood pulp; regenerated cellulosics, such as rayon and cellulose acetate; peat moss, loofa; and the like. One skilled in the art would readily appreciate that a blend of two or more types of absorbent materials may be used to optimize the performance of fluid management articles used in varying conditions. The absorbent material may be uniformly dispersed within the absorbent core structure 50, or may alternatively be placed in discrete patterns, or in gradients. For example, in an effort to help reduce side leakage, absorbent material may be placed in high concentrations around peripheral portions of the core.

The absorbent core structure 50 may have a blend of absorbent materials and thermoplastic fibers, for example to provide structural integrity to the formed structure or for heat sealability to additional layers, such as a barrier layer film. Useful thermoplastic fibers are polyolefins, such as polypropylene and polyethylene fibers, and bicomponent fibers. The thermoplastic fibers may be bi-component or multi-component fibers having a first component having a first melting temperature and two or more additional components having different melting temperatures to that of the first melting temperature. Bi-component fibers are typically configured sheath-core or side-by-side. Suitable bi-component fibers include polyester/polyethylene and polypropylene/polyethylene. In one aspect, the present invention can provide a fluid management article containing at most 12 weight percent of an absorbent material such as cellulose-based material.

The body-facing layer 20 and absorbent core structure 50 may be joined together by co-embossing. For example, embossing two materials with a series of dots may be used to join the materials. The dots will provide additional fluid retaining spaces and may contribute to the overall softness in terms compressibility. One can calculate compressibility as follows: thickness of uncompressed material minus the thickness of the compressed material (2.5 pounds per square inch) multiplied by 100 and dividing the product by the thickness of the uncompressed material. The dots may also aid in providing flexibility to the finished article; the embossed dots may allow the article to bend without surface deformation or wrinkling.

Transfer Layer

Optionally, the absorbent article of the present invention may include a transfer or distribution layer. If included in the absorbent article, the transfer layer may be made of any known material that will take up fluid and then distribute and release it to an adjacent absorbent layer for storage. Transfer layers have a relatively open structure that allows for movement of fluid within the layer. Suitable materials for such transfer layers include fibrous webs, resilient foams, and the like.

A transfer layer is typically between the body-facing layer and the absorbent structure.

The mass of materials making up the transfer layer may be absorbent, although the materials themselves are not absorbent. Thus, transfer layers that are made of hydrophobic, nonabsorbent fibers may be able to accept large volumes of fluid into interfiber void spaces while the fibers themselves do not absorb any significant quantities of fluid. Likewise, open-celled foam structures that are made from nonabsorbent materials may also absorb fluid into the cells of the foam. The walls of the cells, however, do not absorb any fluid. The cumulative spaces within the transfer layer, i.e., the interfiber void spaces in the fibrous transfer layer or the open cells in the foam transfer layer, function much like a container to hold fluid.

Typically, transfer layer fibrous webs are made of resilient, nonabsorbent materials to provide void volume and to allow for free movement of fluid through the structure. Transfer layers that are made from webs of mostly absorbent fibers absorb the fluid as it enters the structure and do not distribute it throughout the rest of the structure as efficiently as webs containing non-absorbent materials.

In those embodiments of the invention where a transfer layer is employed it is possible that indicia 70 may be printed on either surface of the transfer layer as long as such surfaces are “internal surfaces” of the article.

Barrier

The garment-facing layer 30 of the present invention may be pliant and is typically referred to as a backsheet or barrier layer. The exterior of the garment-facing layer 30 forms the garment-facing surface 23 of the absorbent article and, typically, is impermeable to fluids. The garment-facing layer also has an inner surface 44. In one embodiment, the garment-facing layer may be any thin, flexible, fluid impermeable material, such as a polymeric film, e.g., polyethylene, polypropylene, or cellophane, or a normally fluid pervious material that has been treated to be impervious, such as impregnated fluid repellent paper or non-woven material, including non-woven fabric material, or a flexible foam, such as polyurethane or cross-linked polyethylene.

Additionally, the garment-facing layer may be breathable, i.e., permits vapor to transpire. Known materials for this purpose include nonwoven materials and microporous films in which microporosity is created by, inter alia, stretching an oriented film. Single or multiple layers of permeable films, fabrics, melt-blown materials, and combinations thereof that provide a tortuous path, and/or whose surface characteristics provide a liquid surface repellent to the penetration of liquids may also be used to provide a breathable backsheet.

The thickness of the garment-facing layer when formed from a polymeric film typically is about 0.001 inch (0.025 mm) to about 0.002 inch (0.051 mm).

The garment-facing layer 30 may be located adjacent to absorbent structure 50 (shown FIG. 4) and also to the cover 20 (shown FIG. 2), especially if the absorbent core is smaller than the cover and barrier (not shown).

Bonding Methods

The layers of the absorbent article may be, but not necessarily, bonded, e.g., glued or adhered, to the adjacent layer. For example, the underside of the cover 20 may be adhered to the topside of the absorbent core 50. The underside of the absorbent core 50 may be adhered to the topside of the barrier layer 40. Any methods known in the art, such as, fusion bonding, adhesive attachment, or by any other securement means can be used to secure the individual layers together to form the final absorbent article. Included within such methods are coembossing, thermobonding, mechanical bonding, and the like. Fusion bonding includes heat bonding, ultrasonic bonding, and the like.

Adhesive is typically used to attach the layers into a single absorbent article. For example, in one embodiment, the body facing cover 10 is attached to the barrier layer 50 with adhesive NW1023 available from H.B Fuller and Company (St. Paul, Minn.). The adhesive may be applied in any method.

Adhesive may include pressure sensitive adhesive that is applied as strips, swirls, or waves, and the like. As used herein, the term pressure-sensitive adhesive refers to any releasable adhesive or releasable tenacious means. Suitable adhesive compositions, include, for example, water-based pressure-sensitive adhesives such as acrylate adhesives. Alternatively, the adhesive composition may include adhesives based on the following: emulsion or solvent-borne adhesives of natural or synthetic polyisoprene, styrene-butadiene, or polyacrylate, vinyl acetate copolymer or combinations thereof; hot melt adhesives based on suitable block copoylmers—suitable block copolymers for use in the invention include linear or radial co-polymer structures having the formula (A-B)x wherein block A is a polyvinylarene block, block B is a poly(monoalkenyl) block, x denotes the number of polymeric arms, and wherein x is an integer greater than or equal to one. Suitable block A polyvinylarenes include, but are not limited to Polystyrene, Polyalpha-methylstyrene, Polyvinyltoluene, and combinations thereof. Suitable Block B poly(monoalkenyl) blocks include, but are not limited to conjugated diene elastomers such as for example polybutadiene or polyisoprene or hydrogenated elastomers such as ethylene butylene or ethylene propylene or polyisobutylene, or combinations thereof. Commercial examples of these types of block copolymers include Kraton™ elastomers from Shell Chemical Company, Vector™ elastomers from Dexco, Solprene™ from Enichem Elastomers and Stereon™ from Firestone Tire & Rubber Co.; hot melt adhesive based on olefin polymers and copolymers where in the olefin polymer is a terpolymer of ethylene and a comonomers, such as vinyl acetate, acrylic acid, methacrylic acid, ethyl acrylate, methyl acrylate, n-butyl acrylate vinyl silane or maleic anhydride. Commercial examples of these types of polymers include Ateva (polymers from AT plastics), Nucrel (polymers from DuPont), Escor (from Exxon Chemical).

Additionally, the absorbent article may be rolled into a compact structure without the cover surface wrinkling. In one embodiment, upon unrolling and removal of the release paper, the absorbent article does not retain “memory” of being rolled. In other words, the absorbent article lays flat or conforms to the article to which it is placed on, in this instance, the crotch portion of a garment such as underwear.

The absorbent article of the present invention may be applied to the crotch of a garment by placing the garment-facing surface against the inside surface of the crotch of the garment. Various methods of attaching absorbent articles may be used. For example, chemical means, e.g., adhesive, and mechanical attachment means, e.g., clips, laces, ties, and interlocking devices, e.g., snaps, buttons, VELCRO (Velcro USA, Inc., Manchester, N.H.), zipper, and the like are examples of the various options available to the artisan.

Adhesive may be applied to the garment-facing side of the absorbent article. The positioning adhesive may be any adhesive known in the art. As a non-limiting example, pressure sensitive adhesive strips, swirls, or waves may be applied to help maintain the absorbent article in place. As used herein, the term pressure-sensitive adhesive refers to any releasable adhesive, or releasable tenacious means. Suitable adhesive compositions, include, for example, water-based pressure-sensitive adhesives, such as acrylate adhesives. Alternatively, the adhesive composition may include rapid setting thermoplastic “hot melt,” rubber adhesives, two-sided adhesive tape, and the like.

Where positioning adhesive is used on the garment-facing side of the garment-facing layer 30, a release strip may be applied to protect the adhesive on the absorbent article prior to attaching the absorbent article to the crotch. The release strip can be formed from any suitable sheet-like material that adheres with sufficient tenacity to the adhesive to remain in place prior to use but which can be readily removed when the absorbent article is to be used. Optionally, a coating may be applied to release strip to improve the ease of removing the release strip from the adhesive. Any coating capable of achieving this result may be used, e.g., silicone. Because typical release strips have memory properties, it is contemplated that while a release strip can be used with the rolled disposable article of the present invention, the memory properties of the disposable absorbent article of the present invention are separate and distinct from any memory properties of the release paper.

Wings

Wings, also called, among other things, flaps or tabs, may also be part of the absorbent article of the present invention. Wings and their use in sanitary protection articles are described in U.S. Pat. No. 4,687,478 to Van Tilburg; U.S. Pat. No. 4,589,876 also to Van Tilburg, U.S. Pat. No. 4,900,320 to McCoy, and U.S. Pat. No. 4,608,047 to Mattingly. The disclosures of these patents are incorporated herein by reference in their entirety.

As disclosed in the above documents, wings are, generally speaking, flexible and configured to be folded over the edges of the underwear so that the wings are disposed between the edges of the underwear.

In addition, there may be one or a plurality of wings incorporated into the present invention. Where opposed wings are present along the longitudinal edges of the absorbent article of the present invention, such wings may be directly opposed or may be offset from the wings located along the opposite longitudinal edge of the absorbent article.

When present, the wings may preferably be folded inwardly prior to rolling the disposable absorbent article.

The silhouette of absorbent articles of this invention includes those designed to fit garments having conventionally-shaped crotches, e.g., briefs and bikinis. Additionally, absorbent articles of the present invention may also be designed to fit garments having abbreviated crotches including thong, string underwear, G-string, Rio cut, Brazilian cut, etc.

Also contemplated herein include asymmetrical and symmetrical articles having parallel longitudinal edges, dog bone- or peanut-shaped, circular, oval and the like. The silhouette of the disposable absorbent article of the present invention may be configured to be used with conventional underwear or may be configured to conform to thong garments. As used herein, the term thong includes, but is not limited to, thong underwear, thong swimming suit bottom, G-strings, Rio cut underwear, Rio cut swimming suit bottom, Brazilian cut underwear, Brazilian cut swimming suit bottom, and any other garment that exposes the buttocks, having a narrow strip of fabric or a cord that passes between the thighs supported by a waistband, a waist cord, belt or the garment itself. The absorbent article may include other known materials, layers, and additives, such as, foam, net-like material, perfumes, medicaments or pharmaceutical agents, moisturizers, odor control agents, and the like. The absorbent article can optionally be embossed with decorative designs.

The absorbent article 10 may be made from any of the processes known to one of ordinary skill in the art. For example, a continuous length of cover (body-facing layer) material may be laminated to a continuous length of absorbent material. This laminate may then be laminated to a backsheet (garment-facing layer) to form a fluid management article. The absorbent article can then be rolled up and over-wrapped to form a single unitary or individual absorbent article. In another embodiment, the body-facing layer is laminated to the garment-facing layer and then rolled up and over-wrapped.

Absorbent Capacity

The absorbent articles of the present invention are intended to manage low volumes of fluid encountered both menstrually and intermenstrually, i.e, daily. In one embodiment, the absorbent structure has less than 0.1 grams of absorbent fibers, and the absorbent article has an absorbent capacity of 6.0 grams or less, as determined by the following method: the release paper paper is removed from the absorbent article to be tested. The absorbent article is weighed to the nearest 0.001 gram and the weight recorded as the Initial Dry Weight. The absorbent article is immersed into a 1% sodium chloride solution with the body-facing layer facing upward for a total of five minutes. The absorbent article is removed and hung vertically for two minutes. The absorbent article is then weighed to the nearest 0.001 gram; this weight is recorded as Weight I. The absorbent article is hung again for two minutes and then re-weighed. This is recorded as Weight II. The steps are repeated until the difference between the weight readings are less then 0.1 gram. This is recorded as the Final Weight. The calculations is thus:
Absorbent Capacity (G)=Final Weight (g)−Initial Dry Weight (g)

In addition to absorbent capacity, the articles of the present invention are designed to be extremely comfortable and non-obtrusive to a user. The collective design attributes are intended to provide daily confidence without compromise to lifestyle, including activity and clothing. Two variables, which may affect the before mentioned design characteristics, are article caliper, compressibility and flexibility. Preferably the articles have a caliper of 2.0 millimeters or less, and more preferably 1.5 millimeters or less. In one embodiment of this invention, the absorbent article 10 may be drapeable, that is having a flexural resistance of about 40 g. or less as tested by the Modified Circular Bend Test, ASTM 4032-82 and as described in U.S. Ser. No. 10/025,299, Drapeable Absorbent Article, filed Dec. 19, 2001, the contents herewith incorporated in entirety by reference. Preferably, the articles have a flexural resistance of about 40 grams or less, and more preferably about 30 g or less, and most preferably a flexural resistance of 20 grams or less.

### EXAMPLES

L*, a*, and b* values were measured on the absorbent article using a Minolta Chromameter CR-300 (Minolta Corp., Ramsey, N.J.). The colorimeter was calibrated to a white standard (model CR-A43, Minolta Corp., Ramsey, N.J.) and set to the L*, a*, and b* scale. The CR-300 measuring head used a pulsed xenon lamp using a CIE standard illuminant C and was equipped with a glass light-projection tube (model CR-A33f, Minolta Corp.), which used a diffused illumination/0′ viewing angle. The probe head had a diameter of 8 mm.

Measurement of Color Using CR300 Chromameter

1. Initially, the instrument is calibrated in the Yxy color space using the white Minolta Plate.

2. After calibration, change to L*a*b* color space before measuring.

3. L*₂, a*₂, and b*₂, were determined by measuring the unprinted, i.e. white portion, of the barrier of the samples. For the inventive samples the barrier was a white polyethylene film (Swanson Plastics Singapore Pte. Ltd., Lot #010803-030803). L*₂, a*_{2.}, and b*₂, were measured directly from the barrier, i.e. without the cover layer.

4. Place the measuring probe perpendicularly on the colored sample or indicia to be measured. It is noted that the measurement of the colored portion are taken through the layers arranged between the indicia and the external surface being viewed. Thus, for the inventive samples the measurements were taken through the cover layer with the probe arranged above a colored portion, i.e. non-white, of the barrier. If necessary, the indicia may be magnified in order that the measuring probe fit entirely within the indicia. The sample being measured should be measured from the appropriate external surface being viewed. For the values set forth in Table 1 the samples were measured from the external body facing surface of the cover.

5. Take the measurement by pressing the trigger. The instrument needs approximately 5 seconds to recharge between measurements. The light on the measuring head is lit when it is ready to measure. The instrument assigns sequential numbers to each measurement.

6. Record the L*₁, a*₁, b*₁ values and calculate ΔE.

Table 1 shows the ΔE values for various colors of the inventive samples as measured through the cover material of the inventive samples. Percent light transmittance is also set forth in Table 1.

Percent light transmittance for the inventive samples was obtained using the above identified apparatus and measuring the light transmittance of the cover material, i.e. the material(s) arranged between the indicia and the external surface being viewed. Thus for purposes of determining light transmittance, the cover material was placed on the measuring apparatus alone, i.e. without the barrier layer.

For the Comparative Samples, light transmittance was determined by carefully removing the barrier from the layers of the article arranged above the barrier (the “upper layers”). A flat sample (size approximately 2.5″ by 2.5″ square) taken from the upper layers was placed in a round sample holder (approximately 60 mm diameter). Measurements were taken by placing the flat sample in the appropriate measuring ports of the instrument.

TABLE 1

| | Comp. | Comp. | Comp. | Comp. | Inventive | Inventive |
|---|---|---|---|---|---|---|
| | Sample 1 | Sample 2 | Sample 3 | Sample 4 | Sample 1 | Sample 2 |
| % light | 22.90 | 40.32 | 39.22 | 26.10 | 52.00 | 51.6 |
| transmittance | | | | | | |
| Process Yellow | ND | ND | ND | ND | 19.17 | 19.83 |
| Process Magenta | ND | ND | ND | ND | 19.3 | 20.39 |
| Process Cyan | ND | ND | ND | ND | 19.53 | 19.79 |
| Green (360) | ND | ND | ND | ND | 19.53 | 19.79 |
| Yellow (012) | ND | ND | ND | ND | 17.66 | 18.43 |
| Green 376 | ND | ND | ND | ND | 17.14 | 15.23 |
| Pantone 545 | ND | ND | ND | ND | 11.10 | 9.11 |
| Pantone 642C | ND | ND | ND | ND | 9.38 | 7.49 |
| Pantone 663 | ND | ND | ND | ND | 9.33 | ND |

In Table 1 above, ND=Not Detectable

| Sample number | Commercial Product Name (date code) |
|---|---|
| Comparative Sample 1 | Whisper Dry & Puffy Slim (Procter & |
| | Gamble Japan, lot # 030721 11) |
| Comparative Sample 2 | Sofy Colored Liner (Aqua Blue, lot # |
| | 321935062) (the upper two |
| | layers and barrier are tinted blue) |
| Comparative Sample 3 | Sofy Colored Liner (Pink, lot # |
| | 331132062) (the upper two |
| | layers and barrier are tinted pink) |
| Comparative Sample 4 | Sofy Colored Liner (Mocha, lot # |
| | 322018062) (the upper two layers |
| | and barrier are tinted mocha) |

Inventive Sample 1—Hot through air bonded bicomponent polypropylene/polyethylene nonwoven cover (30 gsm) from ES Fiber GuangZhou Co., LTD (Chisso), hot through air bonded nonwoven core rayon/bicomponent polypropylene/polyethylene material (50 gsm) from ES Fiber GuangZhou Co. Ltd (Chisso), white PE film from Swanson Plastics (Singapore) PTE Ltd., the internal surface of the barrier was printed at Great Wall Industries (Malaysia) using Passion Inks from Coates Brothers Malaysia by flexo or gravure printing.
Inventive Sample 2-75 gsm spunlace body facing layer made from 75% polyester and 25% rayon (3P075V25P75 from Spuntech Industries Ltd., Upper Tiberias, Israel) and a white PE film from Swanson Plastics (Singapore) PTE Ltd., the internal surface of the barrier was printed at Great Wall Industries (Malaysia) using Passion Inks from Coates Brothers Malaysia by flexo or gravure printing.

We claim:

1. An absorbent article comprising:
a first layer having an internal surface and an external surface;
a second layer having an internal surface and an external surface;
indicia arranged on at least one of said internal surfaces of said first layer or on said internal surface of said second layer;
wherein at least one of said first layer or said second layer has a light transmittance of at least 45% wherein said indicia is viewable from at least one of said external surfaces.

2. The absorbent article according to claim 1, wherein at least one of said first layer or said second layer has a light transmittance of at least 45% over its entire surface area.

3. The absorbent article according to claim 1, wherein said indicia has a ΔE of at least 9.

4. An absorbent article comprising:
a liquid permeable cover layer having an internal surface and an external surface;
a liquid impermeable barrier layer having an internal surface and an external surface;
indicia arranged on said internal surface of said barrier;
said cover layer having a light transmittance of at least 45% over its entire surface area and said indicia having a ΔE of at least 9 as measured from said external surface of said cover layer.

5. The absorbent article according to claim 4, said barrier layer having a light transmittance of at least 45% over its entire surface area and said indicia having a ΔE of at least 9 as measured from said external surface of said barrier layer.

6. The absorbent article according to claim 4, wherein said article has an absorbent capacity less than 6 grams of fluid.

7. An absorbent article of claim 6, wherein the article has a flexure resistance of less than 40 g.

8. An absorbent article of claim 6, wherein the article has a flexure resistance of less than 30 g.

9. An absorbent article of claim 6, wherein the article has a flexure resistance of less than 20 g.

10. An absorbent article comprising:
a liquid permeable cover layer having an internal surface and an external surface;
a liquid impermeable barrier layer having an external surface;
an internal layer arranged between said cover layer and said barrier layer, said internal layer having a first surface in abutting relationship to said internal surface of said cover;
indicia arranged on said first surface of said internal layer;
said cover layer having a light transmittance of at least 45% over its entire surface area and said indicia having a ΔE of at least 9 as measured from said external surface of said cover layer.

11. The absorbent article according to claim 10, said barrier layer having a light transmittance of at least 45% over its entire surface-area and said indicia having a ΔE of at least 9 as measured from said external surface of said barrier layer.

12. The absorbent article according to claim 11, wherein said article has an absorbent capacity less than 6 grams of fluid.

13. An absorbent article of claim 11, wherein the article has a flexure resistance of less than 40 g.

14. An absorbent article of claim 11, wherein the article has a flexure resistance of less than 30 g.

15. An absorbent article of claim 11, wherein the article has a flexure resistance of less than 20 g.

16. An absorbent article comprising:
a cover layer having an internal surface and a external surface;
a barrier layer having an internal surface and a external surface, said barrier having non-colored portion and a colored portion printed on said internal surface of said barrier layer;
said cover layer having a light transmittance of at least 45% over its entire surface area and said colored portion having a ΔE of at least 9 as measured from said external surface of said cover layer.

17. The absorbent article according to claim 16, wherein said article has a flexure resistance of less than 40 g and an absorbent capacity of less than 6 grams of fluid.

18. An absorbent article comprising:
a liquid permeable cover layer having an internal surface and an external surface;
a liquid impermeable barrier layer having an external surface;
an internal layer arranged between said cover layer and said barrier layer, said internal layer having a first surface in abutting relationship to said internal surface of said cover and a second surface in opposed relationship to said first surface;
indicia arranged on said second surface of said internal layer;
said cover layer and said internal layer having a light transmittance of at least 45% over their entire surface area and said indicia having a ΔE of at least 9 as measured from said external surface of said cover layer.
